(19) United States
(12) Patent Application Publication
(10) Pub. No.: US 2018/0362918 A1
(43) Pub. Date: Dec. 20, 2018

(54) Cell Therapy Processes Utilizing Acoustophoresis

(71) Applicant: FloDesign Sonics, Inc., Wilbraham, MA (US)

(72) Inventors: Bart Lipkens, Bloomfield, CT (US); Rui Tostoes, Northampton, MA (US); Brian Dutra, East Longmeadow, MA (US); Rudolf Gilmanshin, Framingham, MA (US); Jason Dionne, Simsbury, CT (US); Walter M. Presz, JR., Wilbraham, MA (US); Benjamin Ross-Johnsrud, Northampton, MA (US); Goutam Ghoshal, East Grafton, MA (US); Kedar Chitale, Vernon, CT (US)

(21) Appl. No.: 16/010,296

(22) Filed: Jun. 15, 2018

Related U.S. Application Data
(63) Continuation-in-part of application No. 15/916,270, filed on Mar. 8, 2018.
Application No. 15/916,270 is a continuation-in-part of application No. 15/586,116, filed on May 3, 2017.
Application No. 15/586,116 is a continuation-in-part of application No. 15/788,784, filed on Oct. 19, 2017.
(60) Provisional application No. 62/374,910, filed on Aug. 15, 2016.
(60) Provisional application No. 62/359,182, filed on Jul. 6, 2016.
(60) Provisional application No. 62/330,947, filed on May 3, 2016.
(60) Provisional application No. 62/410,312, filed on Oct. 19, 2016.
(60) Provisional application No. 62/468,895, filed on Mar. 8, 2017.
(60) Provisional application No. 62/520,488, filed on Jun. 15, 2017.

(51) Int. Cl. C12M 1/00; A61K 35/12

(52) U.S. Cl. CPC C12M 47/06; B06B 1/0607; A61K 35/12

(57) ABSTRACT

A closed and modular fluidic system composed of one or more acoustic elements and cell processing reagents. The system employs a cellular manufacturing process for producing cell and gene therapy therapeutics.

### CROSS-REFERENCE TO RELATED APPLICATIONS

This application is a continuation-in-part of U.S. patent application Ser. No. 15/916,270, filed on Mar. 8, 2018. This application is also a continuation-in-part of U.S. patent application Ser. No. 15/586,116, filed on May 3, 2017, which claims the benefit of U.S. Provisional Patent Application Ser. No. 62/330,947, filed on May 3, 2016, and U.S. Provisional Patent Application Ser. No. 62/359,182, filed on Jul. 6, 2016, and U.S. Provisional Patent Application Ser. No. 62/374,910, filed on Aug. 15, 2016. This application is also a continuation-in-part of U.S. patent application Ser. No. 15/788,784, filed on Oct. 19, 2017, which claims the benefit of U.S. Provisional Patent Application Ser. No. 62/410,312, filed on Oct. 19, 2016. U.S. patent application Ser. No. 15/916,270, filed on Mar. 8, 2018, also claims the benefit of U.S. Provisional Patent Application Ser. No. 62/468,895, filed on Mar. 8, 2017. This application also claims the benefit of U.S. Provisional Patent Application Ser. No. 62/520,488, filed on Jun. 15, 2017. The entire disclosures of these applications are hereby fully incorporated herein by reference.

### BACKGROUND

Cell therapy is an immunology based therapy for treating a patient using cellular material. Current processes for implementing cell therapy treatments are associated with very high costs, on the order of $500,000-$1.5 million. A number of processes are used to produce the therapeutic product, with each process tending to be independent, open or nonsterile, and implemented by a highly skilled person or persons that often hold PhDs.

### BRIEF SUMMARY

Cell therapy is a therapy that uses cellular material to treat a patient. Such a therapy sometimes involves obtaining cells, which may be provided by the patient, modifying the cells for therapeutic purposes, and introducing the cells into the patient. The production process for obtaining a final product that is introduced to the patient involves a number of steps or processes for handling and/or manipulating the cellular material. The present disclosure discusses a number of such processes that are implemented using acoustics to separate and/or retain and/or filter materials.

In some examples, a system is provided that is a closed and modular fluidic system composed of acoustic elements and cell processing reagents for a cellular manufacturing process on the scale of 30 to 150 billion cells and 750 mL to 5 L.

In some examples, the process steps include mononuclear cell (MNC) isolation from apheresis products, isolation of T-cells (CD3+, CD3+CD4+ and CD3+CD8+) from apheresis products, removal of T-cell receptor positive cells (TCR+ cells) post cell expansion, as well as several wash and volume change steps.

Implementations may include scale-dependent and/or scale-independent applications, or combinations thereof. Example implementations may control the cellular manufacturing process starting and final cell population and/or automate these process steps.

The various example processes may include one or more of the following, which may be independent or integrated or combined in various combinations or sequences. It should be understood that any types of cellular material may be processed with the disclosed acoustic cellular processing systems and methods. The following examples include processes for T-cells, and one or more of the processes may be applied, independently or in various combinations, to other types of cells.

An apheresis product is obtained, which may include a number of particles or components including T-cells, red blood cells (RBCs), platelets and/or granulocytes. The various components are separated, for example, with an acoustic process that differentiates the particles based on size, density, compressibility and/or acoustic contrast factor. In another example, T-cells are separated from the apheresis product using an affinity selection process. The affinity selection process may implement selection based on markers, including CD3+, CD3+CD4+, CD3+CD8+, for example. Another separation example provides label-free selection of mononucleated cells (MNC) from the apheresis product.

An example process provides for activation of the T-cells using a nanobead process in which acoustics are used to retain or pass the activated T-cells. The activated T-cells may be genetically modified with a lentiviral transduction operation, which may be implemented with an acoustic process that traps and/or co-locates the T-cells and lentivirus. The T-cells may be washed and/or concentrated and/or washed, in any desired order or to produce any desired results for concentrate/wash operations, using one or more acoustic devices that can retain the T cells and concentrate them into a reduced volume. The T-cells may be subjected to electroporation. The T-cells population may be expanded, such as by culturing, using an acoustic device that maintains or recycles the T cells in a culture in which the culture media is exchanged. The expanded T-cell population may be washed and/or concentrated and/or washed using one or more acoustic devices that can retain the T cells and concentrate them into a reduced volume. The T-cell culture may be separated to remove TCR+ cells, which may be achieved through negative selection using an affinity process that retains the TCR+ cells using acoustics. The resulting TCR−-CAR+ cells can be recovered using an acoustic process that separates those cells from the host fluid. A fill and finish process can be implemented on the recovered T cells to prepare a dose representing the final product.

In some example systems, a cell volume of about 30 billion cells or less can be processed in a one liter process. In some example systems, a cell volume of about 150 billion cells or less can be processed in a five liter process. In these example systems, the affinity selection of CD3+ T cells from apheresis products is Ficoll-free. In addition, or alternatively, the affinity selection of CD3+, CD3+CD4+ and/or CD3+CD8+, or any other type of marker selection desired, is Ficoll-free.

In some example systems, a concentrate-wash process and affinity selection process is integrated in a single device. The device can be configured to be used in a one or five liter process, or in any process scale desired.

In some example systems, the acoustic separation process for separating the apheresis components is implemented using an acoustic angled wave device. The acoustic angled wave device permits fractionation of different sized particles at different angles with an acoustic wave applied at an angle to a flow direction.

Concentrating therapeutic cells and transferring them from one solution into another (usually referred to as washing) is discussed herein. In particular, therapeutic cells may originally be suspended in a growth serum or in preservative materials like dimethyl sulfoxide (DMSO). Separating the cells from these fluids so the cells can be further processed is important in the overall therapeutic process of using such cellular materials. In one example, the cells are typically recovered from a bioreactor, concentrated, and transferred from culture media into an electroporation buffer prior to transduction, such as in manufacturing CAR-T cells. After expansion of cells at the final manufacturing step, they are concentrated and transferred into an appropriate solvent depending on the desired application.

Therapeutic cells are stored in specialized media to prolong the viability of these cells either through refrigeration and or freezing processes. Such specialized media may not be compatible when the therapeutic cells are introduced into a patient. It may thus be helpful to both wash and concentrate the therapeutic cells in a buffer or wash media that is biocompatible with both the therapeutic cells and with the patient. The washing step may be repeated a number of times. For example, the specialized media (which can be pyrogenic or otherwise harmful) may be fully removed with multiple wash steps, and the cells may be suspended in a new buffer or wash solution.

Separation of biomaterials can be accomplished by functionalized material distributed in a fluid chamber. The functionalized material bind the specific target materials such as recombinant proteins and monoclonal antibodies or cells. The functionalized material, which may take a form of microcarriers that are coated with an affinity protein, is trapped by nodes and/or anti-nodes of an acoustic standing wave. In this approach, the functionalized material is trapped without contact (for example, using mechanical channels, conduits, tweezers, etc.).

The present disclosure provides methods and systems for replacing or augmenting conventional centrifugation and physical filtration processes along with the multiple washing steps with a simpler, lower cost, and more friendly process for particles such as therapeutic cells. The methods/processes can be performed in a sterile/closed environment and in a continuous form.

Disclosed herein are methods of washing particles, which comprise feeding an initial mixture of a first media and the particles through a flow chamber of an acoustophoretic device. For example, the first media may contain preservatives such as dimethyl sulfoxide (DMSO) which are undesirable for future applications/uses of the particles, such as cells. The acoustophoretic device also comprises at least one ultrasonic transducer that includes a piezoelectric material that is configured to be driven to create a multi-dimensional acoustic standing wave in the flow chamber. The at least one ultrasonic transducer is driven to create a multi-dimensional acoustic standing wave in the flow chamber, such that at least a portion of the particles are trapped in the multi-dimensional acoustic standing wave. The trapped particles are subsequently mixed with a second media to wash the trapped particles (e.g. remove the first media from the particles).

In some embodiments, the initial mixture is run through the flow chamber to obtain an intermediate mixture of the particles in a reduced volume of the first media. The intermediate mixture is then collected, and mixed together with the second media to form a secondary mixture. The secondary mixture is then fed through the flow chamber to obtain a final mixture of particles in a reduced volume of the second media.

In other embodiments, the second media is fed into the flow chamber after the initial mixture is fed through the flow chamber. Here, the second media displaces the first media, or gradually replaces the first media. The second media can be a biocompatible wash or a buffer solution.

In still other embodiments, the acoustophoretic device further comprises a collector located below the at least one ultrasonic transducer so that as the trapped particles form clusters and grow to a critical size and subsequently fall out of the multi-dimensional acoustic standing wave, the clusters fall into the collector. The collector leads to a collection container that contains the second media, mixing the clusters of particles together with the second media.

The particles may be cells. The cells may be Chinese hamster ovary (CHO) cells, NSO hybridoma cells, baby hamster kidney (BHK) cells, human cells, regulatory T-cells, Jurkat T-cells, CAR-T cells, B cells, or NK cells, peripheral blood mononuclear cells (PBMCs), algae, plant cells, bacteria, or viruses. The cells may be attached to m icrocarriers.

Sometimes, the piezoelectric material of the at least one ultrasonic transducer is in the form of a piezoelectric array formed from a plurality of piezoelectric elements. Each piezoelectric element can be physically separated from surrounding piezoelectric elements by a potting material. The piezoelectric array can be present on a single crystal, with one or more channels separating the piezoelectric elements from each other. Each piezoelectric element can be individually connected to its own pair of electrodes. The piezoelectric elements can be operated in phase with each other, or operated out of phase with each other. The acoustophoretic device may further comprise a cooling unit for cooling the at least one ultrasonic transducer.

Also disclosed herein are acoustophoretic systems, comprising an acoustophoretic device with a port that may operate as a wash inlet, a concentrate outlet and/or a wash outlet. The acoustophoretic device may include one or more ultrasonic transducers including a piezoelectric material. The piezoelectric material can be excited to form a standing wave on its surface, which can generate a multi-dimensional acoustic standing wave in an adjacent fluid.

### BRIEF DESCRIPTION OF THE DRAWINGS

The following is a brief description of the drawings, which are presented for the purposes of illustrating the example embodiments disclosed herein and not for the purposes of limiting the same.

FIG. 1 is a block diagram of a cell therapy production process.

FIG. 2 is a diagram of an acoustic angled wave process.

FIG. 3 is a diagram illustrating a magnetically activated affinity process.

FIG. 4 is a diagram illustrating an acoustically activated affinity process.

FIG. 5 is a flowchart illustrating a process for depletion of TCR+ cells.

FIG. 6 is a set of diagrams illustrating an acoustic separation process.

FIGS. 7 and 8 are a set of diagrams illustrating a concentrate-wash operation.

FIG. 8 is a conventional single-piece monolithic piezoelectric material used in an ultrasonic transducer.

FIG. 9 is a block diagram illustrating affinity processes.

FIG. 10 is a block diagram illustrating an integrated concentrate-wash-cell selection device.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. Furthermore, it should be understood that the drawings are not to scale.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named components/steps and permit the presence of other components/steps. However, such description should be construed as also describing compositions or processes as “consisting of” and “consisting essentially of” the enumerated components/steps, which allows the presence of only the named components/steps, along with any impurities that might result therefrom, and excludes other components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

A value modified by a term or terms, such as “about” and “substantially,” may not be limited to the precise value specified. The approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, e.g. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, e.g. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, e.g. ground level. The terms “upwards” and “downwards” are also relative to an absolute reference; an upwards flow is always against the gravity of the earth.

The present application refers to “the same order of magnitude.” Two numbers are of the same order of magnitude if the quotient of the larger number divided by the smaller number is a value of at least 1 and less than 10.

Cell Therapy Processes

Cell therapy is a therapy that uses cellular material to treat a patient. Such a therapy sometimes involves obtaining cells, which may be provided by the patient, modifying the cells for therapeutic purposes, and introducing the cells into the patient. The production process for obtaining a final product that is introduced to the patient involves a number of steps or processes for handling and/or manipulating the cellular material. The present disclosure discusses a number of such processes that are implemented using acoustics to separate and/or retain and/or filter materials.

In some examples, a system is provided that is a closed and modular fluidic system composed of acoustic elements and cell processing reagents for a cellular manufacturing process on the scale of 30 to 150 billion cells and 750 mL to 5 L.

In some examples, the process steps include mononuclear cell (MNC) isolation from apheresis products, isolation of T-cells (CD3+, CD3+CD4+ and CD3+CD8+) from apheresis products, removal of T-cell receptor positive cells (TCR+ cells) post cell expansion, as well as several wash and volume change steps.

Implementations may include scale-dependent and/or scale-independent applications, or combinations thereof. Example implementations may control the cellular manufacturing process starting and final cell population and/or automate these process steps.

The various example processes may include one or more of the following, which may be independent or integrated or combined in various combinations or sequences. It should be understood that any types of cellular material may be processed with the disclosed acoustic cellular processing systems and methods. The following examples include processes for T-cells, and one or more of the processes may be applied, independently or in various combinations, to other types of cells.

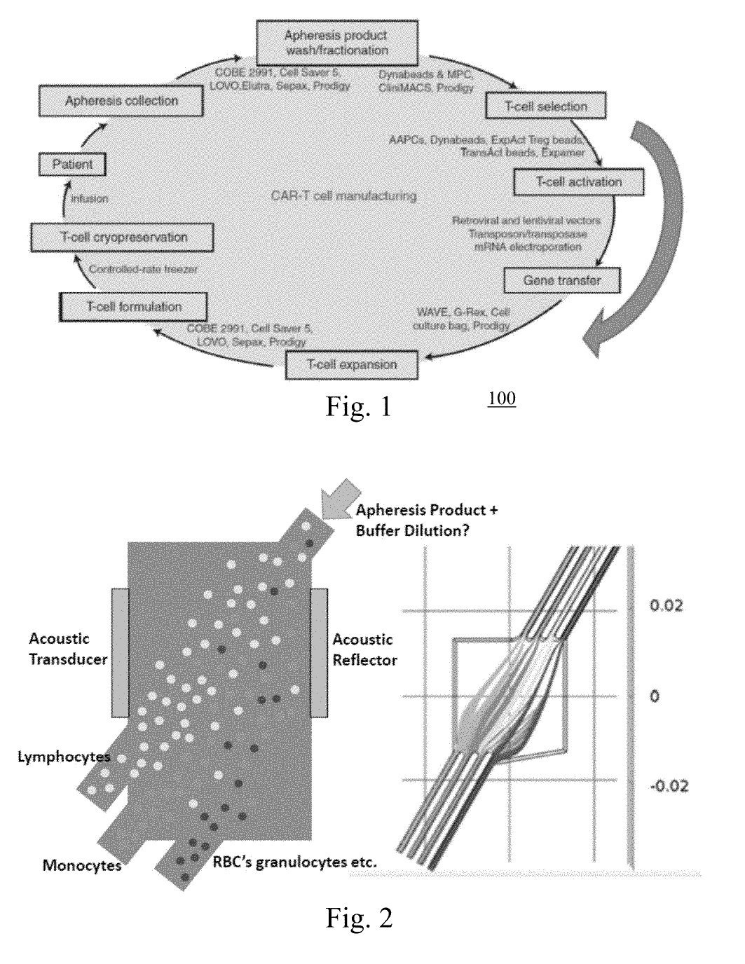

Referring to FIG. 1, a block diagram 100 illustrates various steps in a cell production process. The process is directed to T cells, however, any type of cellular material can be processed with the acoustic devices described herein. The various steps illustrated are apheresis collection, apheresis product wash/fractionation, T-cell selection, T-cell activation, gene transfer, T-cell expansion, T-cell formulation and T-cell cryopreservation. In accordance with the present disclosure, acoustic processing can be applied to some or all of these steps, some of which may be combined or integrated within a single acoustic device.

As illustrated in diagram 100, an apheresis product is obtained, which may include a number of particles or components including T-cells, red blood cells (RBCs), platelets and/or granulocytes. The various components are separated, for example, with an acoustic process that differentiates the particles based on size, density, compressibility and/or acoustic contrast factor. In another example, T-cells are separated from the apheresis product using an affinity selection process. The affinity selection process may implement selection based on markers, including CD3+, CD3+CD4+, CD3+CD8+, for example. Another separation example provides label-free selection of mononucleated cells (MNC) from the apheresis product.

Diagram 100 illustrates activation of the T-cells using a nanobead process in which acoustics are used to retain or pass the activated T-cells. The activated T-cells may be subjected to a gene transfer process, which may involve a lentiviral transduction operation, which may be implemented with an acoustic process that traps and/or co-locates the T-cells and lentivirus. The T-cells may be washed and/or concentrated and/or washed, in any desired order or to produce any desired results for concentrate/wash operations, using one or more acoustic devices that can retain the T cells and concentrate them into a reduced volume. The T-cells population may be expanded, such as by culturing, using an acoustic device that maintains or recycles the T cells in a culture in which the culture media is exchanged. The expanded T-cell population may be washed and/or concentrated and/or washed using one or more acoustic devices that can retain the T cells and concentrate them into a reduced volume. The T-cell culture may be separated to remove TCR+ cells, which may be achieved through negative selection using an affinity process that retains the TCR+ cells using acoustics. The resulting TCR−-CAR+ cells can be recovered using an acoustic process that separates those cells from the host fluid. A fill and finish process can be implemented on the recovered T cells to prepare a dose representing the final product.

Acoustic Angled Wave Separation

RBC depletion and other fractionation processes may be implemented using angled wave technology. The fractionation of RBC, granulocyte, platelet and MNC using the angled wave device is discussed below. FIG. 2 illustrates an acoustic transducer that generates a bulk acoustic wave within a fluid flow with a mean direction flow that is angled relative to the acoustic wave. The angled acoustic wave can cause particles within the fluid to deflect at different angles that depend upon various characteristics of the particles. Thus, bulk acoustic standing waves angled relative to a direction of flow through a device can be used to deflect, collect, differentiate, or fractionate particles or cells from a fluid flowing through the device. FIG. 2 illustrates generation of angled acoustic standing waves due to the acoustic waves being reflected with the acoustic reflector. It should be understood that any type of acoustic wave may be used, including traveling waves, which may be implemented without an acoustic reflector, or maybe implemented with an acoustic absorber. The illustrated acoustic standing wave can be used to separate or fractionate particles in the fluid by, for example, size, density, speed of sound, and/or shape. The angled acoustic standing wave can be a three-dimensional acoustic standing wave. The acoustic standing wave may also be a planar wave where the piezoelectric material of the acoustic transducer is excited in a piston fashion, or the acoustic standing waves may be a combination of the planar acoustic standing waves and the multidimensional acoustic standing waves. The deflection of the particles by the standing wave can also be controlled or amplified by the strength of the acoustic field, the angle of the acoustic field, the properties of the fluid, the dimensionality or mode of the standing wave, the frequency of the standing wave, the acoustic chamber shape, and the mixture flow velocity.

When acoustic standing waves propagate in liquids, the fast oscillations may generate a non-oscillating force on particles suspended in the liquid or on an interface between liquids. This force is known as the acoustic radiation force. The force originates from the non-linearity of the propagating wave. As a result of the non-linearity, the wave is distorted as it propagates and the time-averages are nonzero. By serial expansion (according to perturbation theory), the first non-zero term will be the second-order term, which accounts for the acoustic radiation force. The acoustic radiation force on a particle, or a cell, in a fluid suspension is a function of the difference in radiation pressure on either side of the particle or cell. The physical description of the radiation force is a superposition of the incident wave and a scattered wave, in addition to the effect of the non-rigid particle oscillating with a different speed compared to the surrounding medium thereby radiating a wave.

As illustrated in FIG. 2, an apheresis product is fractionated into lymphocytes, monocytes and RBCs, granulocytes and other particles. This process can be used to isolate T cells in the apheresis product.

Affinity Separation

The affinity separation of biological materials, such as proteins or cells, is accomplished in some examples through the use of a ligand that is covalently bonded to a structure, such as a microbead. The ligand interacts with the protein or cell such that the protein or cell is bound to the ligand on the microbead.

A ligand is a substance that forms a complex with the biomolecules. With protein-ligand binding, the ligand is usually a molecule which produces a signal by binding to a site on a target protein the binding typically results in a change of confirmation of target protein. The ligand can be a small molecule, ion, or protein which binds to the protein material. The relationship between ligand and binding partner is a function of charge, hydrophobicity, and molecular structure. Binding occurs by intermolecular forces such as ionic bonds, hydrogen bonds and van der Waals forces. The Association of docking is actually reversible through disassociation. Measurably irreversible covalent bonds between the ligand and target molecule is a typical in biological systems.

A ligand that can bind to a receptor, alter the function of the receptor, and trigger a physiological response is called an agonist for the receptor. Agonist binding to receptor can be characterized both in terms of how much physiological response can be triggered and in terms of the concentration of the agonist that is required to produce the physiological response. High affinity ligand binding implies that the relatively low concentration of the ligand is adequate to maximally occupy a ligand-binding site and trigger a physiological response. The lower the Ki level is, the more likely there will be a chemical reaction between the pending and the receptive antigen. Low-affinity binding (high Ki level) implies that a relatively high concentration of the ligand is required before the binding site is maximally occupy and the maximum physiological response to the ligand is achieved. Bivalent ligands consist of two connected molecules as ligands, and are used in scientific research to detect receptor timers and to investigate the properties.

The T cell receptor, or TCR, is a molecule found on the surface of T cells or T lymphocytes, that is responsible for recognizing fragments of antigen as peptides bound to major histocompatibility complex (MHC) molecules. The binding between TCR and antigen peptides is of relatively low affinity and is degenerative.

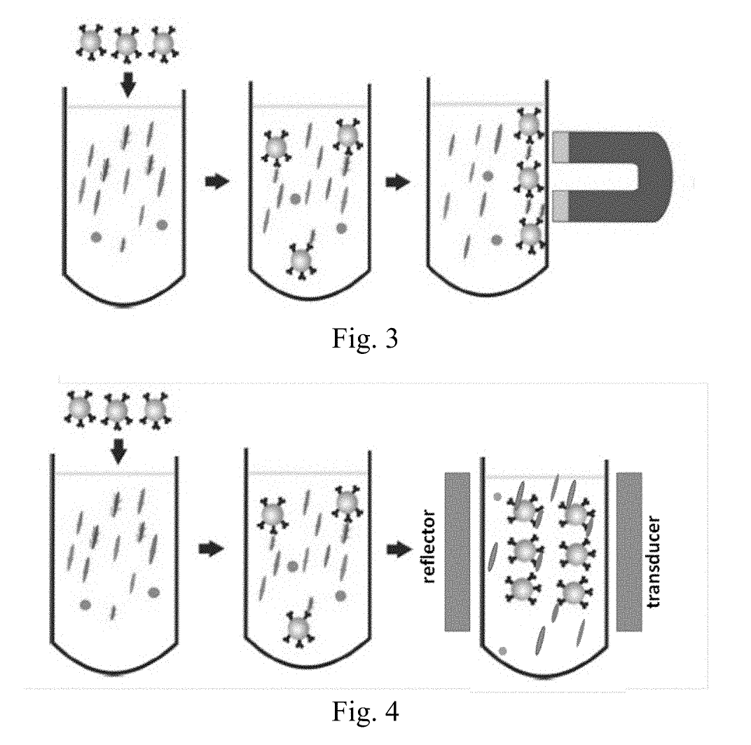

Referring to FIG. 3, paramagnetic beads, such as iron or ferro-magnetic beads sold under the name Dynabeads, have been used to achieve affinity extraction. The magnetic beads, coated with a functionalized material, bind to biological targets in complex mixtures to permit the target material to be separated out of the complex mixture using a magnetic field. The beads carry molecules for affine binding various targets with high specificity. The beads are injected into the complex mixture and incubated to bind the targets. The beads are extracted by a magnet together with the targets attached to the beads.

Micro sized beads are available, such as, e.g., Dynabeads, which are on the order of 4.5 μm in size. Nano sized beads may be used, such as, e.g., Myltenyi, which are on the order of 50 nm in size. Some of the affine molecules that may be used include antibodies, aptamers, oligonucleotides and receptors, among others. The targets for the affinity binding may include biomolecules, cells, exosomes, drugs, etc.

Referring to FIG. 4, beads with high acoustic contrast and affinity chemistry are illustrated. These acoustic beads can be used in exactly the same way as magnetic beads with regard to having functionalized material coatings or composition for affinity binding. The acoustic beads are designed to be extracted from a complex mixture or fluid with an acoustic field. The acoustic beads can be directly used in all the applications developed in cell manufacturing, biochemistry, diagnostics, sensors, etc. that use magnetic beads.

The acoustic beads can use the same surface and affinity chemistry as is used with magnetic beads. This ease of substitution of acoustic beads for magnetic beads has many advantages, including simplifying approval for applications, as well as simplifying the applications.

The acoustic beads can be made biocompatible. Such beads can be produced in different sizes, which permits continuous separation based on size in a size differentiating acoustic field, such as may be provided with an angled-field fractionation technology. The acoustic beads can be combined with an enclosed acoustics-based system, leading to a continuous end-to-end cycle for therapeutic cell manufacturing. This functionality provides an alternative to magnetic bead extraction, while preserving use of currently existing affinity chemistry, which can be directly transferred to the acoustic beads. The acoustic beads may be a consumable product in the separation operation.

In an example, a proof of concept trial was made using the published Memorial Sloan Kettering Cancer Center (MSKCC) protocol for extraction of CD3+ T cells from patient's blood. In the trial, paramagnetic beads were used, and the magnetic field is replaced with an acoustic field. The process of extracting CD3+ T cells from patient's blood is an integral part of manufacturing CAR (chimeric antigen receptor) T cells. Current processes are based on commercially available CD3 Dynabeads. In the trial, efforts were made to minimize the protocol differences, including performing the experiments in culture broth, rather than blood. The difference is considered reduced since several steps in CAR T cell manufacturing work from broth. The solvent density was increased to make T cells “acoustically invisible,” or not as susceptible to an acoustic field. The small size of the Dynabeads may provide an acoustic contrast that is similar to the cells, thus making separation tolerances smaller. The trial employed Jurkat CD3+ and CD3− T cell lines as models. The CD3− cells were employed as a control for non-specific trapping.

The cell suspensions were incubated with CD3 Dynabeads, which bound CD3+ cells. The mixture was passed through the acoustic system, which trapped the magnetic beads (with or without cells). The collected cells were successfully grown in culture. They cultured cells were examined with overlap of bright field images with fluorescence images. The beads were black with slight reddish autofluorescence. The live cells were fluorescent red. The bead diameter is 4.5 microns. CD3+ T-cell complexes with beads were observed, which demonstrates the efficiency of the technique. No CD3− T-cells were extracted in this example, which demonstrates the specificity and selectivity of the technique.

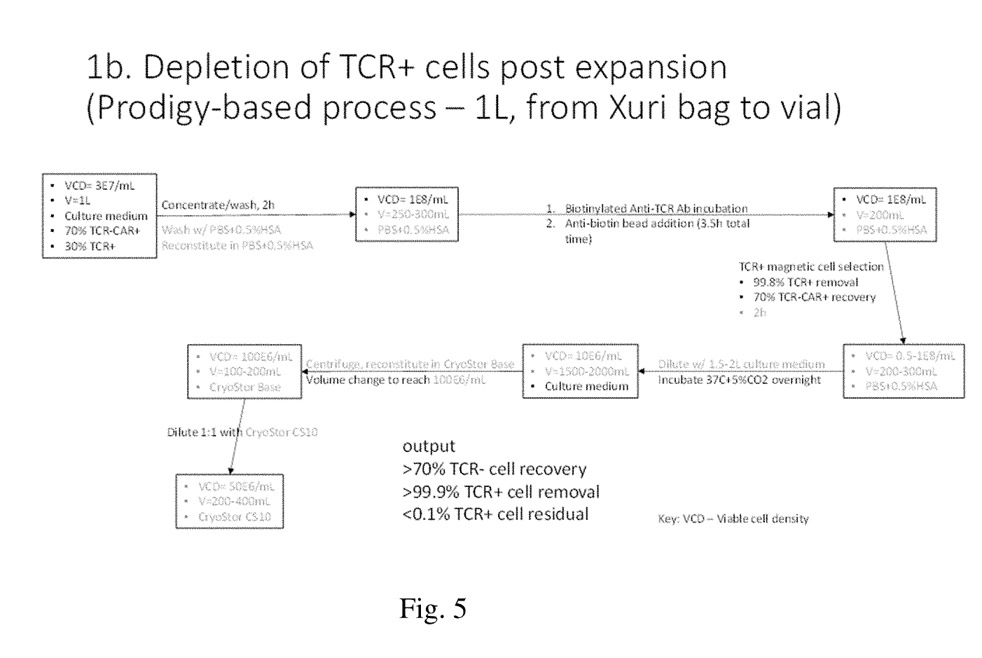

Referring to FIG. 5, a process for affinity selection and removal of TCR+ cells is illustrated. The process steps include a concentrate/wash step, followed by incubation with biotinylated anti-TCR Ab beads. The beads are used to select and remove TCR+ cells through a magnetic process, followed by a culturing and centrifuge process. In accordance with the present disclosure, acoustically sensitive beads are used instead of magnetic selection beads. The acoustic beads may have the same or similar surface chemistry as the magnetic beads. The acoustic beads may be used to select and remove the TCR+ cells has discussed herein.

In an example, a trial with acoustic beads was conducted. In this trial, agarose beads were used as the acoustic beads. These beads are available off-shelf from several manufacturers, and are not paramagnetic or have little to none iron or ferro magnetic content. Some agarose beads have surface modifications that simplify antibody attachment. They are also composed of biocompatible material, which can be important for therapeutic solutions. For example, ABTBeads, which are relatively inexpensive, heterogeneous (20-150 μm), off-shelf beads, which are available with streptavidin and biotin conjugates can be used. CellMosaic agarose beads, which tend to be relatively expensive, homogeneous (20-40 μm) can be configured with any modification by order.

The acoustic beads can be trapped in an acoustic field, such as a multi-dimensional acoustic standing wave. Proof-of-concept and validation of performance has been shown using acoustic affinity beads in an acoustic system. The disclosed methods and systems permit the use of off-shelf reagents, and currently available acoustic systems. The affinities can target any type of desired T cells or markers including TCR+, CD3+, CD4+, CD8+. The acoustic beads can have a high, neutral or low contrast factor, which can affect how the beads respond to an acoustic field, for example being urged toward an acoustic node or antinode, or passing through the field.

The beads may be composed of various materials and combinations, which permits development of optimal chemistry with acoustic performance and biocompatibility. The beads may be processed for isolation, sorting or any other function useful in a separation process. When used with a tuned acoustic system, the performance of specifically designed acoustic beads can match or exceed that of paramagnetic beads.

Existing chemistries may be used with the acoustic beads, and in conjunction with specifications of size and structure homogeneity to achieve desired results for acoustic and for isolation performance. The beads may be composed of composite constructs to advance acoustic efficiency. The acoustic system provides flexibility to manage small sizes, with heat management, and the use of fluidics to obtain results that are not possible with paramagnetic beads alone. The biocompatibility and/or biodegradability of the acoustic beads and simplified processing permits integration with existing hardware for CAR T cell manufacturing. The affinity acoustic beads can be used in a number of environments, including model environments such as, e.g., animal blood spiked with target cells and murine spleen extracts. The acoustic beads may thus be used in collaboration with existing systems, and may be designed and manufactured for target applications. The beads may be provided with a core that is acoustically active or neutral, and the bead themselves may be configured for high, neutral or low acoustic contrast. The size of the beads may be configured for separation and affinity in combination, for example a certain sized bead may include functionalized material to target a certain biomaterial, while another sized bead, may be functionalized to target another biomaterial, each of which can be separated simultaneously and continuously in a closed or flowing system. The beads can be designed to be of a homogeneous size distribution within a narrow or relatively broad range. Various affinity chemistries may be used, including streptavidin-biotin complex and immunoglobulin or aptamer. The beads may be designed for ease of manufacturability and/or for shelf-life. The beads may be used with approved chemistries, so that they may readily be integrated into known systems that use approved chemistries.

Affinity negative selection of TCR+ cells with a volume of 1 L and 30 billion cells was specified in an example trial. In a parallel trial, affinity negative selection of TCR+ cells with a volume of 5 L and 150 billion cells was specified. Table 1 summarizes the results for the trials.

TABLE 1

| | | |
|---|---|---|
| Item | Baseline | Preferred |

| | | |
|---|---|---|
| Initial volume (flexible if | 1 L | (5 L) |
| FDS owns previous stage | | |
| of the process) | | |
| Final volume | 100-200 mL | (500-1000 mL) |
| Total viable cells | 30B | (150B) |

| | | |
|---|---|---|
| Viable TCR⁻CAR⁺ cell | 70% | >70% |
| recovery | | |
| TCR⁺ cell removal efficiency | 99.9% | >99.9% |

Affinity selection of CD3+ cells from an apheresis product was specified in an example trial. Table 2 summarizes the results for the trial.

TABLE 2

| Item | Baseline | Preferred |
|---|---|---|
| Initial volume | 300 mL | |
| Final volume | To be adjusted for activation | |
| Total viable cells | 15B MNCs (correct if T-cells) | |
| Viable CD3⁺ cell recovery | 80% | >80% |
| Purity | 95% CD3⁺ | >95% |

Affinity selection of CD3+CD4+ and CD3+CD8+ cells from an apheresis product was specified in an example trial. Table 3 summarizes the results for the trial.

TABLE 3

| | | |
|---|---|---|
| Item | Baseline | Preferred |

| | | |
|---|---|---|
| Initial volume | 300 | mL |

| | |
|---|---|
| Final volume | To be adjusted for activation |

| | | |
|---|---|---|
| Total viable cells | 15B | MNCs |

| | | |
|---|---|---|
| Viable CD3+CD4+ | 80% | >80% |
| and CD3+CD8+ cell | | |
| recovery | | |
| Purity | 95% CD3+CD4+ and | >95% |
| | CD3+CD8+ | |

Label-free selection of mononucleated cells (MNC) from apheresis product was specified in an example trial. Table 4 summarizes the results for the trial.

TABLE 4

| Requirement | Baseline | Preferred |
|---|---|---|
| Initial volume | 300 mL | |
| Final volume | To be adjusted for activation | |
| Total viable cells | 15B MNCs (correct if T-cells) | |
| Viable MNC recovery | 80% | >80% |
| RBC, Platelets and | 99% | >99% |
| Granulocyte removal | | |
| efficiency | | |

The MNCs were generated by incubating washed and FICOLL treated aspheresis product with beads.

Tests demonstrated the effectiveness of using a fluidized bed device with 30 or 60 μm PLURIBEADS, 34 and 90 μm 6% cross-linked agarose beads, 200 μm STREAMLINE beads, and/or 50 μm POROS beads. Cells bound to nanobeads such as MILTENYL beads did not have enough acoustic contrast for differentiation effective differentiation by the fluidized bed device. Some of these tests demonstrated used a fluidized bed device similar to the device described in more detail in U.S. patent application Ser. No. 15/963,809 filed on Apr. 26, 2018 which is incorporated herein by reference in its entirety.

Concentrate/Wash

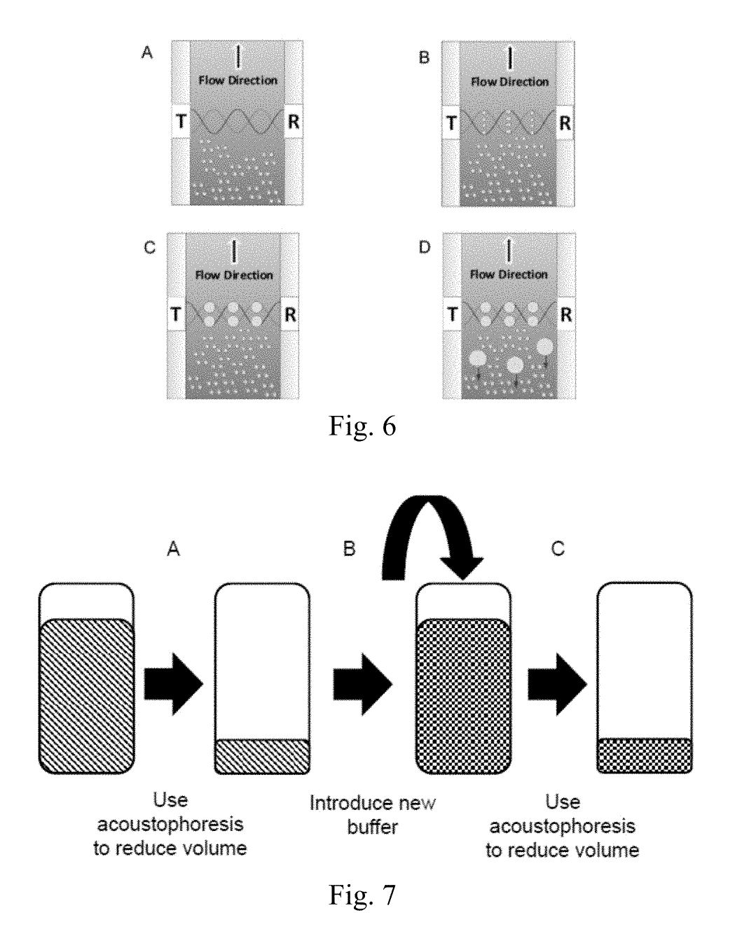

The acoustophoretic technology of the present disclosure employs acoustic standing waves to concentrate, wash, and/or separate materials (such as particles or a secondary fluid) in a primary or host fluid. In particular, as shown in the upper left image (A) of FIG. 6, an ultrasonic transducer T creates an acoustic wave in the fluid, which interacts with a reflector R positioned across from the ultrasonic transducer to create an acoustic standing wave. Although a reflector R is illustrated in FIG. 6, another transducer may be used to reflect and/or generate acoustic energy to form the acoustic standing wave.

As shown in the upper right image (B) of FIG. 6, as the host fluid and material entrained in the host fluid flows upwards through the acoustic standing wave, the acoustic standing wave(s) traps (retains or holds) the material (e.g., secondary phase materials, including fluids and/or particles). The scattering of the acoustic field off the material results in a three-dimensional acoustic radiation force, which acts as a three-dimensional trapping field.

The three-dimensional acoustic radiation force generated in conjunction with an ultrasonic standing wave is referred to in the present disclosure as a three-dimensional or multi-dimensional standing wave. The acoustic radiation force is proportional to the particle volume (e.g. the cube of the radius) of the material when the particle is small relative to the wavelength. The acoustic radiation force is proportional to frequency and the acoustic contrast factor. The acoustic radiation force scales with acoustic energy (e.g. the square of the acoustic pressure amplitude). For harmonic excitation, the sinusoidal spatial variation of the force drives the particles to the stable positions within the standing waves. When the acoustic radiation force exerted on the particles is stronger than the combined effect of fluid drag force and buoyancy and gravitational force, the particle can be trapped within the acoustic standing wave field, as shown in the upper right image (B) of FIG. 6.

As can be seen in the lower left image (C) of FIG. 6, this trapping results in coalescing, clumping, aggregating, agglomerating, and/or clustering of the trapped particles. Additionally, secondary inter-particle forces, such as Bjerkness forces, aid in particle agglomeration.

As the particles continue to coalesce, clump, aggregate, agglomerate, and/or cluster the particles can grow to a certain size at which gravitational forces on the particle cluster overcome the acoustic radiation force. At such size, the particle cluster can fall out of the acoustic standing wave, as shown in the lower right image (D) of FIG. 6.

Desirably, the ultrasonic transducer(s) generate a three-dimensional or multi-dimensional acoustic standing wave in the fluid that exerts a lateral force on the suspended particles to accompany the axial force so as to increase the particle trapping capabilities of the standing wave. A planar or one-dimensional acoustic standing wave may provide acoustic forces in the axial or wave propagation direction. The lateral force in planar or one-dimensional acoustic wave generation may be two orders of magnitude smaller than the axial force. The multi-dimensional acoustic standing wave may provide a lateral force that is significantly greater than that of the planar acoustic standing wave. For example, the lateral force may be of the same order of magnitude as the axial force in the multi-dimensional acoustic standing wave.

The acoustic standing waves of the present disclosure can be used to trap particles (e.g. therapeutic cells such as T cells, B cells, NK cells) suspended in a first media in the standing wave. The first media can then be replaced with a second media (e.g., a biocompatible wash or buffer solution). Put another way, the host fluid of the particles can be replaced. Prior to replacing the first media with the second media, acoustophoresis can be used to perform a diafiltration process, as shown in FIG. 7.

In FIG. 7, starting with an initial mixture that has a low cell density of, for example, less than 1×10⁶ cells/mL, acoustophoresis can be used to reduce the volume of the initial mixture, for example by at least 10×, including 20× and up to 100× or more. The cell concentration may be increased by at least 10×, including 20× and up to 100× or more. This initial reduction process is the first volume reduction step (A). Next, the second media (e.g., a biocompatible wash or buffer solution) can be introduced to at least partially displace the first media, as indicated in step (B). Next, the new mixture of the cells and second media can be subjected to an acoustophoretic volume reduction step (C). This series of operations is referred to as a “diafiltration” process.

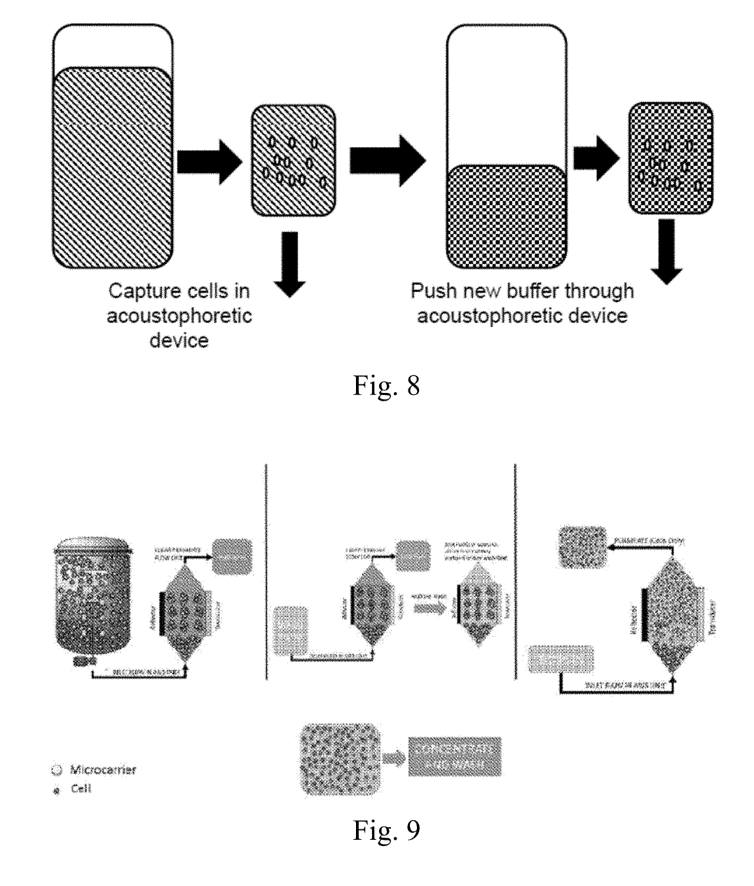

FIG. 8 illustrates a single-step, push-through process in which particles/cells are trapped in the acoustic standing wave and held in the acoustophoretic device. The second media (e.g., a biocompatible wash or buffer solution) is then flowed into the acoustophoretic device to effectively “wash out” the first media. With the push-through process, more than 90%, including up to 99% or more, of the first media can be removed from the particles/cells. The push-through process can be employed as a continuous, single-use process that uses less buffer solution and less time than the diafiltration process of FIG. 7.

The piezoelectric transducer(s) of the acoustophoretic devices and systems of the present disclosure can be single monolithic piezoelectric materials or can be made from an array of piezoelectric materials. The piezoelectric material can be a ceramic material, a crystal or a polycrystal, such as PZT-8 (lead zirconate titanate). The outer surface and the inner surface are relatively large in area, and the crystal is relatively thin (e.g. about 0.040 inches for a 2 MHz crystal).

Each piezoelectric element in the piezoelectric array of the present disclosure may have individual electrical attachments (e.g. electrodes), so that each piezoelectric element can be individually controlled for frequency and power. These elements can share a common ground electrode. This configuration allows for not only the generation of a multi-dimensional acoustic standing wave, but also improved control of the acoustic standing wave. In this way, it is possible to drive individual piezoelectric elements (or multiple, separate ultrasonic transducers) with arbitrary phasing and/or different or variable frequencies and/or in various out-of-phase modes.

The concentration efficiency of the acoustophoretic device was tested. First, a T-cell suspension having a cell density of 1×10⁶ cells/mL was used. A feed volume of between about 500 and 1000 mL was used at a flow rate of 10-15 mL/minute. The device exhibited a concentration factor of between 10× and 20×, a 90% cell recovery, and a 77% washout efficiency (e.g., the amount of the first media that was displaced by the second media) over ten minutes of testing. A 10° C. temperature increase was observed.

The concentration efficiency of the acoustophoretic device was again tested with a higher cell density. A T-cell suspension having a cell density of 5×106 cells/mL was used. A feed volume of 1000 mL was used at a flow rate of 10-15 mL/minute. The device exhibited a concentration factor of better than 10×, a 90% cell recovery, and a 77% washout efficiency over one hour of testing. A 10° C. temperature increase was again observed.

The concentration efficiency of an acoustophoretic device with a 0.75 cubic inches element volume (i.e., the volume between the transducer and the reflector) was tested using Jurkat T-cells. A T-cell suspension having a cell density of 1×10⁶ cells/mL with an initial viability of 94% was used. A feed volume of between about 2,000 mL was used at a flow rate of 15 mL/minute. The device was operated at 30 W. The acoustic capacity of the device was 7.5×10⁸ where acoustic capacity is the total number of cells that can be held/trapped between transducer and reflector in an acoustic system. The acoustic capacity of a system is a function of the volume between transducer and reflector, the frequency and power at which the system is being operated, and the number of nodes. No washing step was used. The results are presented in Table 5.

TABLE 5

| | | Concentration | |
|---|---|---|---|
| | Volume (mL) | (viable cells/mL) | Viability |
| Processed Feed | 2,057 | 1.13 × 10⁶ | 94% |
| Concentrate | 25.5 | 69.9 × 10⁶ | 82% |
| Waste | 1,775 | 0.153 × 10⁶ | 94% |
| Feed left in | 202 | 0.982 × 10⁶ | 94% |
| filtration bag | | | |

Based on these results, the device demonstrated an 84% viable cell recovery (i.e., viable cells in/viable cells out).

The concentration efficiency of the acoustophoretic device was also tested using Primary T-cells. A feed volume of about 1,000 mL was used at a flow rate of 15 mL/minute. The device was operated at 30 W. The acoustic capacity of the device was 7.5×10⁸. The concentration step was followed by a 50 mL wash using an electroporation buffer. Three runs were performed with the results presented in Table 6, Table 7, and Table 8, respectively.

TABLE 6

| | Volume | Concentration | | Viable |
|---|---|---|---|---|
| | (mL) | (viable cells/mL) | Viability | Cells |
| Wash | 65.3 | | | |
| Feed | 1,027.4 | 0.99 × 10⁶ | 96% | 1.02 × 10⁹ |
| Waste | 1,054.7 | 0.161 × 10⁶ | 92.5% | 0.17 × 10⁹ |
| Concentrate | 18.6 | 43.5 × 10⁶ | 96.7% | 0.81 × 10⁹ |

Based on the results for the first run, the device demonstrated a retention rate (i.e., cells not in waste) of 83% and a recovery rate (i.e., cells actually recovered) of 80%.

TABLE 7

| | Volume | Concentration | | Viable |
|---|---|---|---|---|
| | (mL) | (viable cells/mL) | Viability | Cells |
| Wash | 56.5 | | | |
| Feed | 1,008.4 | 1.04 × 10⁶ | 94.6% | 1.05 × 10⁹ |
| Waste | 1,039.3 | 0.127 × 10⁶ | 93.9% | 0.13 × 10⁹ |
| Concentrate | 19.1 | 42.3 × 10⁶ | 95.7% | 0.81 × 10⁹ |

Based on the results for the second run, the device demonstrated a retention rate (i.e., cells not in waste) of 87% and a recovery rate (i.e., cells actually recovered) of 77%.

TABLE 8

| | Volume | Concentration | | Viable |
|---|---|---|---|---|
| | (mL) | (viable cells/mL) | Viability | Cells |
| Wash | 59.2 | | | |
| Feed | 986.7 | 0.98 × 10⁶ | 93.6% | 0.97 × 10⁹ |
| Waste | 1,005.5 | 0.196 × 10⁶ | 94.1 | 0.20 × 10⁹ |
| Concentrate | 24.4 | 31.1 × 10⁶ | 96.5% | 0.76 × 10⁹ |

Based on the results for the third run, the device demonstrated a retention rate (i.e., cells not in waste) of 80% and a recovery rate (i.e., cells actually recovered) of 78%.

A fourth run was performed later using Primary T-cells. A feed volume of about 1,000 mL was used at a flow rate of 15 mL/minute. The device was operated at 30 W. The acoustic capacity of the device was 7.5×10⁸ cells. The concentration step was followed by a 50 mL wash using an electroporation buffer. Approximately 18M TCR+ cells were captured in the column with a maximum purity (i.e., the highest negative selection of the TCR+ cells, leaving the target cells, TCR−, to flow through the column) achieved of 99% and a maximum recovery of TCR− cells achieved of 80%.

A fifth run was performed using Primary T-cells in an acoustophoretic device with a 3.68 cubic inches element volume. A feed volume of about 1,000 mL was used at a flow rate of 15 mL/minute. The device was operated at 30 W. The acoustic capacity of the device was 36.8×10⁸. The concentration step was followed by a 50 mL wash using an electroporation buffer. Approximately 450M TCR+ cells were captured in the column with a maximum purity achieved of 95% and a maximum recovery of TCR− cells achieved of 95%.

Three capacity experiments were also run using the same feed. In the first capacity experiment, the feed was processed through a flow chamber device with a 0.75 cubic inches element volume at flow rate of 15 ml/min and a power of 30 W, without any dropout or recirculation steps and the waste line was monitored every 2 minutes. The goal of this experiment was to determine the maximum number of cells this acoustic element could hold before significant breakthrough of cells to the waste line (capacity). The waste line VCD values increased from 14 to 57% of the feed concentration during the 20 minutes run. The final VCD in the concentrate was 152E6/ml at a viability of 93.5% and the volume was 18.33 mL.

In the second capacity experiment, the feed was processed through a 5 L flow chamber device with a 3.68 cubic inches element volume at a flow rate of 30 ml/min and a power of 77 W. The acoustic capacity of the device was 36.5×10⁸. The waste line VCD increased from 3% to 63% of the feed concentration during the 40 minute run. The final VCD in the concentrate was 117E6/ml at a viability of 95.9% and the final volume was 73.65 ml.

The third capacity experiment was identical to the second capacity experiment except that the flow rate was doubled to 60 ml/min that was expected to lead to an earlier breakthrough. The waste line VCD increased from 27% to 62% of the feed concentration throughout the 16 minute run. The final VCD in the concentrate was 80.7E6/ml at 97.4% viability and the concentrate volume was 81.1 mL.

During testing, it was also discovered that active cooling of the ultrasonic transducer led to greater throughput and efficiency and more power. As such, a cooling unit was developed for actively cooling the transducer. The cooling unit includes an independent flow path that is separate from the flow path through the device containing the fluid that is to be exposed to the multi-dimensional acoustic standing wave. A coolant inlet is adapted to permit the ingress of a cooling fluid into the cooling unit. A coolant outlet serves as the outlet through which the coolant and waste heat exit the cooling unit. Here, the coolant inlet is located below the coolant outlet, though this path can be varied as desired. The coolant that flows through the cooling unit can be any appropriate fluid. For example, the coolant can be water, air, alcohol, ethanol, ammonia, or some combination thereof. The coolant can, in certain embodiments, be a liquid, gas, or gel. The coolant can be an electrically non-conductive fluid to prevent electric short-circuits. The cooling unit can be used to cool the ultrasonic transducer, which can be particularly advantageously when the device is to be run continuously with repeated processing and recirculation for an extended period of time (e.g., perfusion). The cooling unit can also be used to cool the host fluid running through the device, if desired.

FIG. 9 illustrates a four-step process (with an optional fifth step) for concentrating, washing, and separating microcarriers from cells. The first step in the process involves concentrating the microcarriers with attached cells in an acoustophoretic device, such as those described herein. The microcarriers and attached cells can be introduced to the acoustophoretic device by receiving the microcarriers with attached cells from a bioreactor. In the bioreactor, the microcarriers and cells are suspended in a first media (e.g., growth serum or preservative material used to keep the cells viable in the bioreactor). The microcarriers with attached cells surrounded by the first media are concentrated by the acoustic standing wave(s) generated in the acoustophoretic device. In a second step, the concentrated microcarriers with attached cells are then washed with a second media to remove the first media (e.g., bioreactor growth serum or preservative material). The third step is to then introduce a third media containing an enzyme into the acoustophoretic device to detach the cells from the microcarriers through enzymatic action of the second media. In particular embodiments, trypsin is the enzyme used to enzymatically detach the cells from the microcarriers. The multi-dimensional acoustic standing wave can then be used to separate the cells from the microcarriers. Usually, this is done by trapping the microcarriers in the multi-dimensional acoustic standing wave, while the detached cells pass through with the third media. However, the cells can be trapped instead, if desired. Finally, the separated cells may optionally be concentrated and washed again, as desired.

After being concentrated and trapped/held in the multi-dimensional acoustic standing wave, the microcarriers can coalesce, clump, aggregate, agglomerate, and/or cluster to a critical size at which point the microcarriers fall out of the acoustic standing wave due to enhanced gravitational settling. The microcarriers can fall into a collector of the acoustophoretic device located below the acoustic standing wave, to be removed from the flow chamber.

During testing, steps one and two of concentration and washing, respectively, were performed using red and blue food dye to make colored fluid. The concentration mixture included SoloHill microcarriers in red fluid. The wash mixture included blue fluid and was passed through the device three times. The concentrate was observed under a microscope. The concentration step was shown to have a 99% efficiency. The first media (dyed red) was progressively washed out by a second media (dyed blue) over a series of wash passes. The light absorbance data is shown in Table 9 below.

TABLE 9

Light Absorbance

| Sample | Red (510 nm) | Blue (630 nm) |
|---|---|---|
| Feed | 0.138 | 0.041 |
| Wash Pass 1 | 0.080 | 0.066 |
| Wash Pass 2 | 0.063 | 0.080 |
| Wash Pass 3 | 0.054 | 0.084 |

The decrease in red light absorbance and increase in blue light absorbance evidences the feasibility of the washing steps. The testing of the acoustophoretic concentrating, washing, and separating process showed that the process is appropriate for cell therapy and microcarrier applications. The concentrate and wash steps were performed with a resulting efficiency of greater than 99%, and the separating step e.g., separating the cells from the microcarriers, was performed with greater than 98% efficiency.

In an example implementation, a concentrate-wash process was employed with a volume of 750 mL, 1.5 billion cells, prior to electroporation. A parallel example implementation had a volume of 5 L and 150 billion cells prior to electroporation. Table 10 summarizes the results for each example.

TABLE 10

| | | |
|---|---|---|
| Item | Baseline | Preferred |

| | | |
|---|---|---|
| Initial volume | 750 mL | (3.75 L) |
| Final volume | 10-25 mL | (50-125 mL) |
| Total viable cells | 1-1.5B | (5-7.5B) |

| | | |
|---|---|---|
| Viable cell recovery | 80% | >80% |

In an example implementation, a concentrate-wash process was employed with a volume of 1 L, 30 billion cells, post cell expansion. A parallel example implementation had a volume of 5 L and 150 billion cells post cell expansion. Table 7 summarizes the results for each of these examples.

TABLE 11

| | | |
|---|---|---|
| Item | Baseline | Preferred |

| | | |
|---|---|---|
| Initial volume | 1 L | (5 L) |
| Final volume (flexible | 100-200 mL | (500-1000 mL) |
| if FDS owns next stage | | |
| of the process) | | |
| Total viable cells | 30B | (150B) |

| | | |
|---|---|---|
| Viable cell recovery | 80% | >90% |

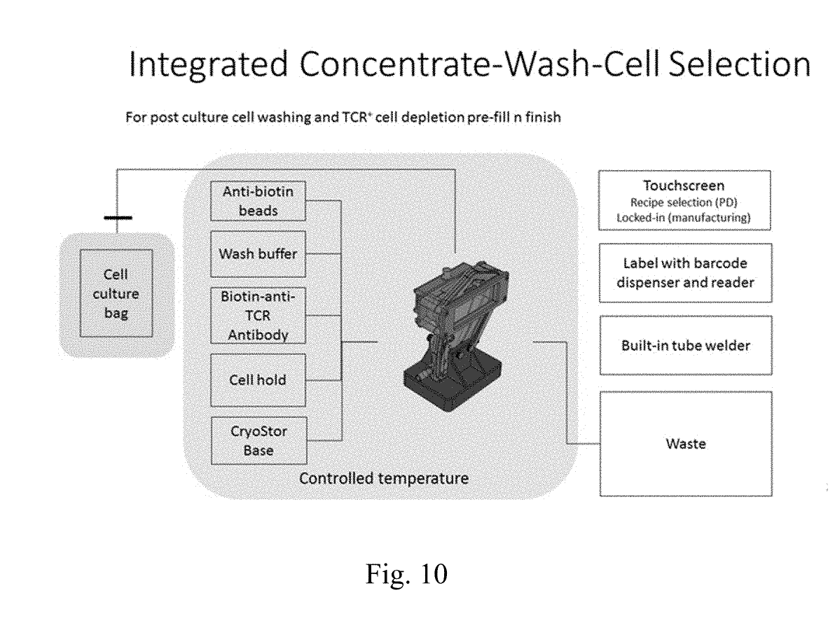

As discussed above, one or more processes in the systems for production of cell therapy products may be integrated in a single device. Referring to FIG. 10, a block diagram of a device suitable for implementing a concentrate-wash process and a cell selection process is illustrated. The illustrated device is capable of mixing and separation operations. A cell culture bag can be loaded into the device for the application of various processes. The cell culture bag includes various ports for fluidic input and/or output. The device provides an acoustic field that can retain cells and/or particles such as beads to implement an affinity selection process, a concentration process and/or a wash process. In some examples, mechanisms are provided to control the inputs, outputs and operations of the device to permit one or more processes to be automated. The automation implementation includes a controller that can operate pumps, valves, ultrasonic transducers, and other equipment used to implement the above noted processes. The automation implementation includes a user interface that's displays information related to various processes, and can accept input for a selection of parameters and/or process steps. The user interface may also provide statistical or process status data.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. It is intended that the present disclosure be construed as including all such modifications and alterations insofar as they come within the scope of the appended claims or the equivalents thereof.

1. A method for producing a therapeutic by implementing a series of processes, the method comprising:
obtaining cellular material suitable for invoking a therapeutic response;
wherein the processes include one or more of a process to concentrate the cellular material, a process to wash the cellular material, or a process for affinity selection of a portion of the cellular material; and
at least one of the processes employing an acoustic device to retain the cellular material or a structure to which the cellular material is bound.

2. The method of claim 1, further comprising:
fractionating the cellular material or the modified cellular material with an acoustic angled wave device.

3. The method of claim 2, wherein the cellular material is included in an apheresis product.

4. The method of claim 1, further comprising integrating one or more of the processes into a single device.

5. The method of claim 1, wherein the cellular material is housed in a bag.

6. The method of claim 1, wherein the series of processes form a closed system.

7. The method of claim 1, wherein the process for affinity selection includes negative selection for TCR+ cells.

8. The method of claim 1, wherein the series of processes form an end-to-end CAR T production process.

9. A system for producing a therapeutic by implementing a series of processes, the system comprising:
an acoustic device that includes an ultrasonic transducer configured to generate an acoustic wave to retain cellular material or a structure to which the cellular material is bound;
a chamber in the acoustic device for receiving the cellular material or a structure to which the cellular material is bound, the ultrasonic transducer being coupled to the chamber;
the acoustic device being configured to implement one or more of a concentration process, a washing process, or an affinity selection process.

10. The system of claim 9, further comprising an angled wave acoustic device for fractionating the cellular material.

11. The system of claim 10, wherein the cellular material is included in an apheresis product.

12. The system of claim 9, wherein the acoustic device is configured to integrate one or more of the concentration process, the washing process, or the affinity selection process.

13. The system of claim 9, wherein the cellular material is housed in a bag.

14. The system of claim 9, further comprising a closed system.

15. The system of claim 9, wherein the affinity selection process includes negative selection for TCR+ cells.

16. The system of claim 9, further comprising an end-to-end CART production process.

17. A cell therapy production system, comprising:
a number of interconnected devices that form a closed system, at least one of the devices being an acoustic device configured to retain cells or structures for supporting cells.

18. The system of claim 17, wherein the devices form and end-to-end cell therapy production system.
